(19) United States
(12) Patent Application Publication
(10) Pub. No.: US 2009/0264535 A1
(43) Pub. Date: Oct. 22, 2009

(54) LIQUID DRUG FORMULATION

(75) Inventors: Venkata-Rangarao Kanikanti, Leverkusen (DE); Gerald Beddies, Overland Park, KS (US); Georg Schulte, Wuppertal (DE)

Correspondence Address: BAYER HEALTHCARE LLC, P.O.BOX 390, SHAWNEE MISSION, KS, 66201, US

(73) Assignee: BAYER ANIMAL HEALTH GMBH, Leverkusen (DE)

(21) Appl. No.: 12/299,127

(22) PCT Filed: Apr. 19, 2007
(86) PCT No.: PCT/EP07/03425

§ 371 Date: Mar. 5, 2009

(30) Foreign Application Priority Data
May 2, 2006 (DE) 10 200 020 604.5

(51) Int. Cl. A61K 31/138; A61P 9/00

(52) U.S. Cl. 514/652

(57) ABSTRACT

The invention relates to a liquid drug formulation for beta-blockers, which is suitable in particular for oral application in animals.

The invention relates to a liquid drug formulation for beta-blockers, which is suitable in particular for oral application in animals.

Beta-blockers (also called beta-receptor blockers), such as bisoprolol, carvedilol and atenolol for example, have been known for a long time in human medicine for the treatment of high blood pressure and, in recent times, cardiac insufficiency. Use of beta-blockers in veterinary medicine is also being considered.

U.S. Pat. No. 5,484,776 describes a method for production of controlled-release formulations of beta-blockers which are suitable for oral application. In this method the beta-blocker is converted with a polysaccharide, preferably xanthan, in water, usually at elevated temperatures.

WO 99/16417 describes aerosol sprays and soft gelatin capsules for oral application. According to the description the formulations described are suitable for a broad spectrum of active ingredients.

WO 03/041696 discloses preparations containing enriched (S)-bisoprolol, and the application thereof for the treatment of cardiovascular disorders.

The requirements for drug formulations in veterinary medicine are especially high, in particular in the case of oral application, since they must have sufficient palatability, so that the animal absorbs the whole dose. As a rule beta-blockers are given in the case of chronic indications, so that the treatment can last for months or years. Furthermore the body weight of the animals treated (e.g. dogs or cats) varies, so that the possibility of variable dosage is also desirable. There is therefore a need for formulations for beta-blockers which combine high acceptance by the animal, good dosage variation and good long-term stability.

The problem is solved by:

Liquid drug formulation on an aqueous basis for oral administration, containing not more than 1% by weight of a beta-blocker in dissolved form, with the formulation exhibiting rapid bio-availability.

The active ingredient group of the beta-blockers is well known to the person skilled in the art. Examples of beta-blockers are: carvedilol, atenolol, acebutolol, propanolol, pindolol, metoprolol, betaxolol, esmolol, nebivolol and bisoprolol.

There are various subgroups of beta-blockers, such as beta-1-selective, beta-2-selective and non-selective, for example. Beta-1-selective beta-blockers, such as atenolol, acebutolol, betaxolol, esmolol, metoprolol, nebivolol and, in particular, bisoprolol, for example, are particularly suitable within the scope of this invention.

Because of their high efficacy the beta-blockers are only used in low concentrations in the formulation according to the invention, usually in concentrations of not more than 1% by weight, preferably not more than 0.5% by weight. The usual concentration ranges for the beta-blockers are therefore 0.001 to 1% by weight, preferably 0.005 to 0.5% by weight, and especially preferably 0.01 to 0.5% by weight.

“On an aqueous basis” means that the formulations according to the invention contain water as an essential solvent, usually at least 40% by weight, preferably at least 50% by weight, especially preferably at least 70% by weight, and more especially preferably at least 80% by weight.

Apart from water the formulation according to the invention can if necessary contain other suitable water-miscible solvents.

For the application of the drug formulation according to the invention it is as a rule desirable that it should be slightly viscous. For this reason the drug formulations according to the invention preferably contain a water-soluble/water-miscible thickener, e.g. glycerine or preferably water-soluble cellulose derivatives such as hydroxypropyl cellulose or hydroxypropyl methylcellulose, for example. The necessary thickener concentrations for production of a formulation with suitable viscosity are known in principle. Thus gelling agents, such as the water-soluble cellulose derivatives for example, are usually contained in concentrations of 1 to 10% by weight, preferably 1 to 5% by weight. If the thickener is a water-miscible solvent, such as glycerine for example, higher concentrations of 1 to 70% by weight, preferably 1 to 60% by weight, are also conceivable.

The solutions preferably have a viscosity of 2 to 20 cP, preferably 4 to 15 cP, especially preferably 5 to 10 cP.

In order to improve palatability the drug formulations according to the invention can contain tastants and/or flavourings. Examples are sugar (usual concentration: 2 to 10% by weight, preferably 3 to 8% by weight) and vanilla flavour (usual concentration: 0.05 to 0.3% by weight, preferably 0.1 to 0.2% by weight). Sweeteners, such as aspartame, cyclamate, saccharin, acesulfame, sucralose, thaumatin, neohesperidin, etc., can also be used. The concentrations of the various sweeteners to be recommended vary; they are generally known to the person skilled in the art, however. Of the sweeteners, saccharin, in particular the sodium salt, is preferred. It is usually employed in a concentration of 0.01-0.5% by weight, preferably 0.02-0.3% by weight.

In order to ensure the long-term stability, the use of preservatives is to be recommended. The preservatives are preferably chosen in such a way that they act against bacteria and fungi. Examples of preservatives are organic acids, such as for example p-hydroxybenzoic acid ester, sorbic acid, benzoic acid, propionic acid, or the salts thereof; alcohols, such as for example benzyl alcohol, butanol or ethanol, and quaternary ammonium compounds, such as for example benzalkonium chloride. An example of an especially suitable preservative is sodium benzoate. The preservative is usually contained in the preparations according to the invention in a quantity of 0.01 to 1% by weight, preferably 0.02 to 0.6% by weight, and especially preferably 0.02 to 0.4% by weight, relative to the total weight of the preparation.

It may furthermore be expedient to adjust the aqueous solution by the addition of suitable buffer substances to a defined pH value, usually in the range 2 to 10, preferably 3 to 9.

Particularly when sodium benzoate is used as a preservative, weakly acidic pH values in the range from 3 to 7, in particular 3 to 5, are preferred.

In addition the drug formulations according to the invention can contain other usual pharmaceutical adjuvants and additives. Other active ingredients, which improve the effect or broaden the spectrum of activity to other indications, can also conceivably be added to the formulations in addition to the beta-blocker.

The drugs according to the invention exhibit rapid bio-availability. They are accordingly characterized in vitro by rapid release kinetics, i.e. at least 75% of the active ingredient is released within 30 minutes (for the method of measurement see “Dissolution”, “Apparatus 2” in US Pharmacopeia 29 [2006]).

The rapid bio-availability can be described in vivo by the attainment of the maximum plasma concentration (Cₘₐₓ) of the active ingredient. This should be attained within 2 hours, preferably 1.5 hours.

Apart from a rapid bio-availability, a high bio-availability is also aimed at; that means that a high proportion of the active ingredient gets into the blood plasma and to the desired point of action, and is not for example directly excreted because it is not absorbed, nor becomes ineffective as a result of metabolization. The formulations according to the invention also exhibit good bio-availability when administered orally, which is as a rule comparable with the bio-availability when administered intravenously.

In the case of low dosages, in particular, a linear (so-called “dose linearity”) and precise correlation between the quantity of active ingredient administered and the resultant plasma concentration should also be achieved, in order to make it possible to give the appropriate dose.

Since the formulations according to the invention are as a rule administered regularly (e.g. daily) over prolonged periods, they should also provide the possibility of repeated, precisely dosed application over a prolonged period.

The drug formulations according to the invention can be produced by mixing the individual components in the necessary quantities. This can be done, for example, by presenting part of the solvent, adding the other components, adjusting the pH value if necessary, and then making up to the required final volume with further solvent. Temperatures above +40° C., preferably above +30° C. are preferably avoided in the production.

The drug preparations according to the invention are generally suitable for application in man and animals. They are preferably employed in animal husbandry and animal breeding in farm animals, animals for breeding, zoo animals, laboratory animals, experimental animals, and pets and hobby animals.

The drug formulations according to the invention are usually employed for the treatment of cardiovascular diseases in animals, and in particular in the treatment of cardiac insufficiency.

The farm animals and animals for breeding include mammals, such as cattle, horses, sheep, pigs, goats, camels, water buffalo, donkeys, rabbits, fallow deer, reindeer, fur-bearing animals such as mink, chinchilla, racoons, and also birds, such as chickens, geese, turkeys, ducks, pigeons, and species of birds intended to be kept in the home and in zoos.

Laboratory and experimental animals include mice, rats, guinea pigs, golden hamsters, dogs and cats.

The pets and hobby animals include rabbits, hamsters, guinea pigs, mice, horses, reptiles, corresponding species of birds, dogs and cats.

The preparations according to the invention are preferably employed in pets and hobby animals such as horses, cats and dogs. They are particularly suitable for application in cats and especially dogs.

Examples of preferred farm animals are cattle, sheep, pigs and chickens.

The formulations here described are intended preferably for oral application.

### EXAMPLES

The formulations can be produced by dissolving all the components except the bisoprolol compound in a quantity of phosphate buffer which is somewhat less than the desired final volume. The bisoprolol compound is then dissolved in the mixture, the pH value is adjusted and the volume is made up to the final volume with phosphate buffer.

### Example 1

0.008% by weight of bisoprolol hemifumarate,

0.20% by weight of sodium benzoate,

0.20% by weight of sodium propionate,

0.15% by weight of vanilla flavour,

5.00% by weight of sugar,

4.00% by weight of HPM cellulose 5 cP

ad 100% by weight of phosphate buffer pH 4.0

### Example 2

0.05% by weight of bisoprolol hemifumarate,

0.2% by weight of sodium benzoate,

0.20% by weight of vanilla flavour,

2.00% by weight of HPM cellulose 5 cP

ad 100% by weight of phosphate buffer pH 4.0

### Example 3

0.40% by weight of bisoprolol hemifumarate,

0.20% by weight of sodium benzoate,

0.15% by weight of vanilla flavour,

2.00% by weight of HPM cellulose 5 cP

ad 100% by weight of phosphate buffer pH 4.0

### Example 4

0.02% by weight of bisoprolol hemifumarate,

0.20% by weight of sodium benzoate,

0.20% by weight of sodium propionate,

0.15% by weight of vanilla flavour,

2.00% by weight of HPM cellulose 5 cP

ad 100% by weight of phosphate buffer pH 4.0

### Example 5

0.005% by weight of bisoprolol hemifumarate,

0.20% by weight of sodium benzoate,

0.20% by weight of sodium propionate,

0.15% by weight of vanilla flavour,

5.00% by weight of HPM cellulose 5 cP

ad 100% by weight of phosphate buffer pH 4.0

### Example 6

0.02% by weight of bisoprolol hemifumarate,

0.14% by weight of 4-hydroxybenzoic acid methyl ester (methylparaben),

0.02% by weight of 4-hydroxybenzoic acid propyl ester (propylparaben),

0.02% by weight of butylhydroxyanisol,

50% by weight of glycerine,

0.25% by weight of vanilla flavour

ad 100% by weight of phosphate buffer pH 6.5

### Example 7

0.02% by weight of bisoprolol hemifumarate,

0.30% by weight of sodium benzoate,

0.15% by weight of vanilla flavour,

2.00% by weight of HPM cellulose 5 cP

ad 100% by weight of phosphate buffer pH 4.0

### Example 8

0.02% by weight of metoprolol tartrate,

0.30% by weight of sodium benzoate,

0.15% by weight of vanilla flavour,

2.00% by weight of HPM cellulose 5 cP

ad 100% by weight of phosphate buffer pH 4.0

### Example 9

0.02% by weight of bisoprolol hemifumarate,

0.20% by weight of sodium benzoate,

0.20% by weight of sodium propionate,

0.15% by weight of vanilla flavour,

5.00% by weight of sugar,

2.00% by weight of HPM cellulose 5 cP

ad 100% by weight of phosphate buffer pH 4.0

### Example 10

0.005% by weight of bisoprolol hemifumarate,

0.05% by weight of sodium benzoate,

0.15% by weight of vanilla flavour,

2.00% by weight of HPM cellulose 5 cP

ad 100% by weight of phosphate buffer pH 4.0

### Example 11

0.01% by weight of bisoprolol hemifumarate,

0.075% by weight of sodium benzoate,

0.15% by weight of saccharin sodium salt,

2.00% by weight of HPM cellulose 5 cP

ad 100% by weight of phosphate buffer pH 4.0

### Example 12

0.08% by weight of bisoprolol hemifumarate,

0.075% by weight of sodium benzoate,

0.15% by weight of saccharin sodium salt,

2.00% by weight of HPM cellulose 5 cP

ad 100% by weight of phosphate buffer pH 4.0

### Example 13

0.33% by weight of bisoprolol hemifumarate,

0.075% by weight of sodium benzoate,

0.15% by weight of saccharin sodium salt,

2.00% by weight of HPM cellulose 5 cP

ad 100% by weight of phosphate buffer pH 4.0

### Example 14

0.05% by weight of bisoprolol hemifumarate,

0.3% by weight of sodium benzoate,

0.15% by weight of vanilla flavour,

0.05% by weight of saccharin sodium salt,

2.00% by weight of HPM cellulose 5 cP

ad 100% by weight of phosphate buffer pH 4.0

### Biological Examples

### A. Pharmacokinetic Investigations

A study was carried out with a total of 18 adult dogs, 6 per group. The test substance was administered to the dogs orally on one occasion in dosages of 0.01 mg/kg, 0.05 mg/kg and 0.1 mg/kg of body weight. Blood samples of about 4 ml were taken after administration of the active ingredient, at the following times: 15, 30, 45, 60, 90 minutes, 2, 4, 6, 8, 12 and 24 hours after administration of the active ingredient.

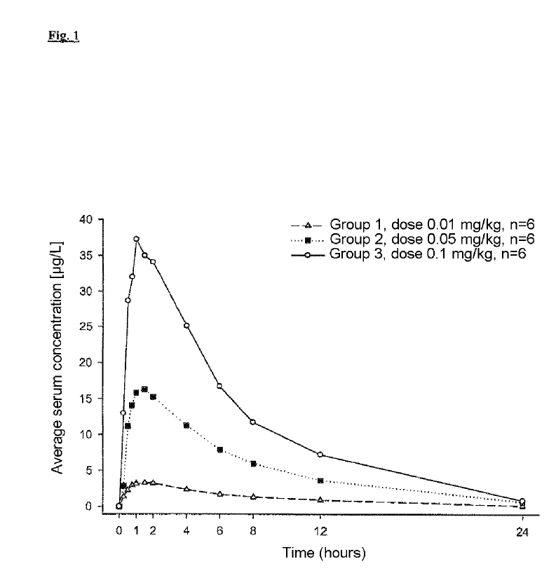

The results with the formulation of Example 6 are shown graphically in FIG. 1. The mean serum concentration of bisoprolol (in μg/L) is plotted against time (in hours). The three curves show the variation in serum concentration for different dosages. Dosage Group 1: 0.01 mg/kg bisoprolol; Group 2: 0.05 mg/kg bisoprolol; Group 3: 0.1 mg/kg bisoprolol.

### B. Comparison of Bio-Availability Oral Versus Intravenous Administration

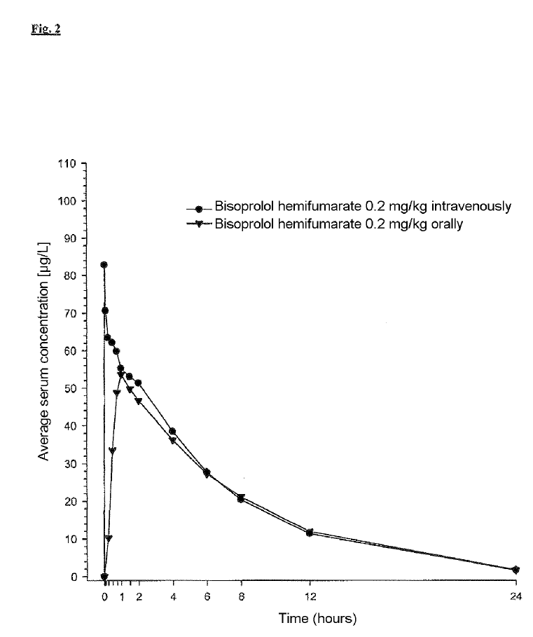

In a further study with 24 dogs, bisoprolol hemifumarate at 0.2 mg/kg of body weight was administered orally (formulation as in Example 14) to 12 dogs and intravenously to 12 dogs. The bisoprolol level in plasma was determined at various times after administration. The results are shown in FIG. 2, where the mean serum concentrations in μg/L are plotted against time in hours. It is found that with oral administration an unusually high bio-availability is achieved, which is almost as high as with direct intravenous application.

1. A liquid drug formulation on an aqueous basis for oral administration, containing not more than 1% by weight of a beta-blocker in dissolved form, with the formulation exhibiting rapid bio-availability.

2. The drug formulation according to claim 1, containing not more than 0.5% by weight of a beta-blocker.

3. The drug formulation according to claim 1, wherein the beta-blocker is bisoprolol.

4. The drug formulation according claim 1, further comprising a water-soluble thickener.

5. The drug formulation according to claim 1, further comprising one or more tastants and/or flavourings.

6. The drug formulation according to claim 4, wherein the water-soluble thickener is a gelling agent.

7. The drug formulation according to claim 6, comprising 1 to 10% by weight of the gelling agent.

8. The drug formulation according to either of claims 6, wherein the gelling agent is a water-soluble cellulose derivative.

9. The drug formulation according to claim 8, wherein the water-soluble cellulose derivative is hydroxypropyl cellulose.

10. The drug formulation according to claim 8, wherein the water-soluble cellulose derivative is hydroxypropylmethyl cellulose.

11. (canceled)
